(19) United States
(12) Patent Application Publication
(10) Pub. No.: US 2019/0107521 A1
(43) Pub. Date: Apr. 11, 2019

(54) SYSTEM AND METHOD FOR FIELD TEST MANAGEMENT

(71) Applicant: AgriSight, Inc., Ann Arbor, MI (US)

(72) Inventors: Devin Riley, Ann Arbor, MI (US); Michael Asher, Ann Arbor, MI (US); Tracy Blackmer, Ann Arbor, MI (US)

(21) Appl. No.: 16/146,113

(22) Filed: Sep. 28, 2018

Related U.S. Application Data
(60) Provisional application No. 62/569,003, filed on Oct. 6, 2017.

(51) Int. Cl. G01N 33/24; G01W 1/14; G01K 3/14

(52) U.S. Cl. CPC G01N 33/24; G01N 2033/245; G01K 3/14; G01W 1/14

(57) ABSTRACT

A method for guided agronomic trial management includes: identifying a field test for a geographic region; and determining field characteristics for the field test based on field data (e.g., current field data, historic field data, etc.) associated with the field test.

### CROSS-REFERENCE TO RELATED APPLICATIONS

This application claims the benefit of U.S. Provisional Application No. 62/569,003 filed 6 Oct. 2017, which is incorporated in its entirety by this reference.

### TECHNICAL FIELD

This invention relates generally to the agricultural field, and more specifically to a new and useful system and method for field test management in the agricultural field.

### BACKGROUND

Agronomic evaluations of plants, fertilizers, and treatment strategies, such as weed or pest control strategies, can be useful to understand the physiography of the system being studied, the growth of the plant, the performance of the fertilizer, or the efficacy of the treatment strategy. In these evaluations, the particular set of climate (e.g., rainfall, temperature), soil type, aspect, use (e.g., grazing practices), management conditions, and other influences on the expected performance can vary from site to site, none of which can be easily controlled. As such, it can be desirable to select a variety of different sites for similar trials. Similarly, it can be desirable to sample from a variety of different sampling points from each trial to determine how each trial does over time.

However, conventionally, the parameters for these trials (e.g., locations, treatment methods, sampling times and locations, etc.) have been determined randomly or based on farmer availability. This leads to: the need for large sample sizes (e.g., many trial locations are required), low trial statistical power, low test robustness, sampling inefficiencies (e.g., more sampled plants are needed), and other drawbacks.

Thus, there is a need in the agricultural field to create a new and useful guided agronomic trial parameter determination and analysis. This invention provides such new and useful guided agronomic trial system and method.

### BRIEF DESCRIPTION OF THE FIGURES

FIGS. 1A-1B are schematic representations of embodiments of a method for test field characterization.

FIG. 2 is a schematic representation of an embodiment of a system for test field characterization.

FIG. 3 is a schematic representation of an embodiment of a system for test field characterization.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

### 1. Overview.

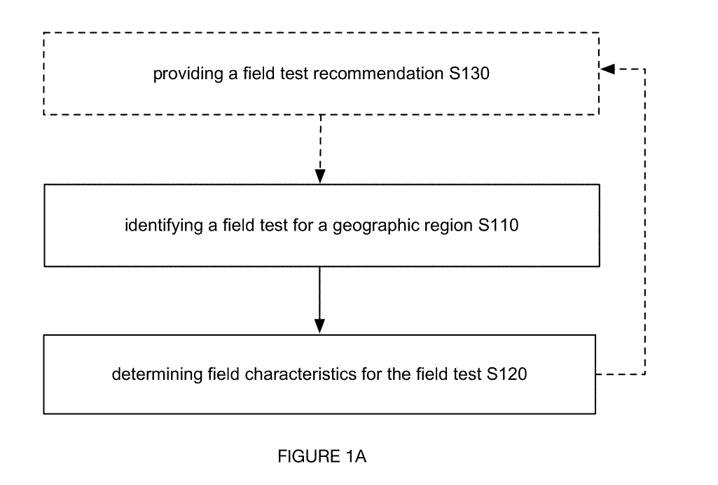

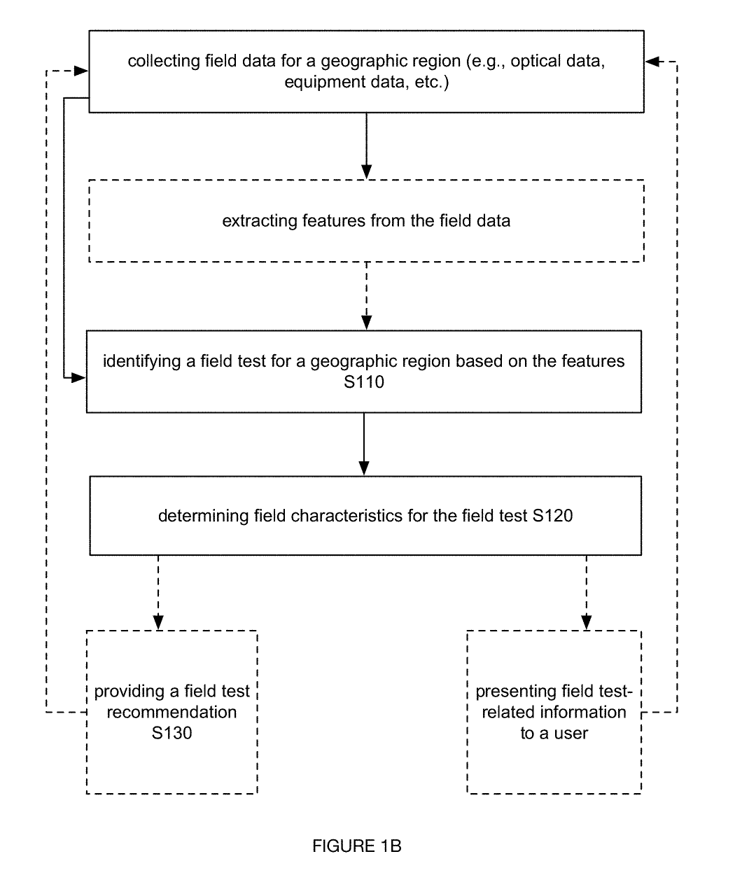

As shown in FIGS. 1A-1B, embodiments of a method 100 for characterizing field tests can include: identifying a field test for a geographic region S110; and determining field characteristics for the field test based on field data (e.g., current field data, historic field data, etc.) associated with the field test S120. Additionally or alternatively, the method 100 can include providing field test recommendations S130; and/or any other suitable processes.

In a specific example, the method 100 can include: collecting first field data (e.g., equipment data, satellite image data, user-provided data, etc.) for a geographic region (e.g., a field, sub-fields of a field, etc.) during a time period; identifying a field test (e.g., detecting the presence of an agronomic trial; identifying location parameters associated with test strips of an agronomic trial; etc.) based on the field data (e.g., equipment sensor data indicative of field tests; treatment information indicating field tests; field patterns determined from image data and representative of field tests; etc.), where the field test is associated with the time period (e.g., ongoing during the time period; scheduled for the time period; etc.); collecting second field data (e.g., equipment data, image data, weather data, soil data, etc.) for the geographic region during a second time period; and determining field characteristics (e.g., yield, test strip performance comparisons, field test robustness, classifications of manmade causes versus natural causes, recommendations, crop anomalies, etc.) associated with the field test for the geographic region based on the field data (e.g., first and/or second field data; historic field data leveraged to account for background variability in evaluating test strip performance; etc.). In another specific example, the method 100 can include: collecting field data (e.g., for a geographic region associated with a user; for geographic regions associated with a population of users; historic field data; etc.) associated with a first time period; determining a field test recommendation (e.g., field test location parameters; field test timing parameters; sampling parameters; etc.) for a user; collecting field data for a second time period associated with a field test (e.g., in response to identifying the field test, etc.) corresponding to the field test recommendation (e.g., test strips established according to the field test recommendation, etc.); and determining field characteristics associated with the field test based on the field data for the second time period.

Embodiments of the method 100 and/or system 200 can function to automatically identify and/or characterize field tests (e.g., agronomic trials). Additionally or alternatively, embodiments can function to prescribe personalized field test recommendations, such as agronomic trial parameters (e.g., placement of on-farm agronomic trials; which crops to plant in which location; test site dimensions or size; test volume; treatment parameters such as treatment type, treatment volume or duration, treatment frequency, etc.; grazing practices; sampling points; sampling frequency; sampling volumes; yield baselines for test yield comparison; etc.) to users for guiding field tests, and/or embodiments can have any suitable function and application. In a first application, field characteristics (and/or other suitable data described herein) for field tests can be presented (e.g., at a web interface; in a report; through automatically generated figures and/or suitable representations of the characteristics; etc.) to a user associated with the geographic region corresponding to the field test, in order to aid the user in optimizing farm management activities (e.g., modifying tillage operations, spraying operations, seeding operations, equipment operations, other suitable operations based on insights extracted from the characterizations for the field test, etc.). In a second application, field test recommendations can be provided for guiding the configuration (e.g., placement, hybrid assignment to test site, treatment parameter, etc.), operation (e.g., sampling), and/or other parameters of field tests in facilitating improvement of the field tests (e.g., in relation to efficiency, statistical power, value, cost, etc.). In a third application, field characteristics identified for field tests can include manmade versus natural variables affecting the field (e.g., where recommendations can be determined and provided to overcome manmade causes of suboptimal productivity, etc.), crop anomalies, improved prescriptions (e.g., fertilizer prescriptions), and/or any other suitable of characteristics facilitating improvement of fields. In a fourth application, field characteristics and/or field test recommendations can be leveraged in a cost (e.g., expense) tracking and/or planning tool for improving value derived from field tests. In a fifth application, the method can analyze or determine the trial results on a per-trial basis or multi-trial basis (e.g., wherein the multiple trials can be for the same growing season, same time point, multiple growing seasons, etc.).

One or more instances and/or portions of the method and/or processes described herein can be performed asynchronously (e.g., sequentially), concurrently (e.g., in parallel; concurrently on different threads for parallel computing to improve system processing ability for processing remotely sensed data, extracting features, characterizing a plurality of field tests, determining a plurality of field test recommendations including different sets of recommended field test parameters for achieving different target parameters, etc.), in temporal relation to a trigger event (e.g., receiving an updated set of remotely sensed data for a geographic region; identifying a field test; receiving updated equipment data; automatically or manually performing the portion of the method in response to the trigger event; etc.), and/or in any other suitable order at any suitable time and frequency (e.g., for the same or different geographic regions, etc.) by and/or using one or more instances of the system, components, and/or entities described herein.

Data described herein (e.g., field characteristics, field data, field test recommendations, features, modules, etc.) can be associated with any suitable temporal indicators (e.g., seconds, minutes, hours, days, weeks, etc.) including one or more: temporal indicators indicating when the data was collected, determined, transmitted, received, and/or otherwise processed (e.g., indicating when remotely sensed optical data was captured for a geographic region; indicating when a field test was established; etc.); temporal indicators providing context to content described by the data, such as time periods in which calculated yield characteristics are relevant (e.g., field test yield corresponding to the end of growing season; in-season yield for a field test; etc.); changes in temporal indicators (e.g., data over time; change in data; data patterns; data trends; data extrapolation and/or other prediction; etc.); and/or any other suitable indicators related to time. However, the method and/or system can be configured in any suitable manner.

### 2. Benefits

In specific examples, the method and system can confer several benefits over conventional approaches. First, the technology can provide technical solutions necessarily rooted in computer technology (e.g., computationally processing aerial satellite imagery, non-generalized farm equipment sensor data, and/or other suitable data to extract features for identifying, characterizing, and/or recommending field tests; computationally refining digital modules with specialized datasets in an iterative process; etc.) to overcome issues specifically arising with the computer technology (e.g., improving accuracy for identification, characterization, and/or recommendation of field tests; overcoming issues associated with accounting for background variability in extracting accurate insights from field tests; improving associated processing speeds for analyzing a plurality of field tests across a plurality of geographic regions, locations, and users; etc.).

Second, the technology can confer an improvement to the functioning of farm management systems (e.g., fertilizer application equipment, seeding machinery, sprayer equipment, planters, tractors, harvesting equipment, etc.). For example, field characterizations extracted for field tests can be presented to a user for facilitating identification of correlations between farm management activities (e.g., associated with different parameters tested in field tests, etc.) and deficiencies in field characteristics (e.g., yield, nutrient status, etc.), where such actionable insights can lead to modifying usage of farm management systems (e.g., automatically, manually, etc.) to overcome the deficiencies in the field characteristics.

Third, the technology can effect a transformation of an object to a different state or thing. Farm management systems can be activated and controlled according to control instructions determined, for example, for configuring, operating, sampling in relation to, and/or performing other suitable processes in relation to field tests. Additionally or alternatively, farm management systems can be activated and controlled for improving geographic regions based on the insights extracted from field tests (e.g., modifying fertilizer prescriptions for a geographic region based on field tests conducted on a geographic sub-region of the geographic region). Remotely sensed digital imagery of a physical field and/or equipment data associated with the physical field can be processed (e.g., through suitable computer vision techniques; through data processing techniques for sensor data; etc.) and transformed. Crop fields can be transformed (e.g., for improving associated yield; etc.) in response to modifications to farm management system operation.

Fourth, the technology can leverage specialized, non-generic systems, such as fertilizer application equipment, seeding machinery, sprayer equipment, planters, tractors, harvesting equipment, and/or other suitable farm management systems, in performing non-generic functions described herein (e.g., providing real-time characterizations of field tests), and/or in performing any suitable functions.

Fifth, the technology can include an inventive distribution of functionality across a network for personalizing content delivered to different users (e.g., personalized to field characteristics for a geographic region associated with the user; personalized to user preferences associated with field tests; etc.). In a specific example, the method 100 can include providing different levels of field characterization based on the types and/or amount of data available for the field test and/or associated geographic region (e.g., comparing yield to equipment operation parameters based on processing equipment data; comparing yield to nitrogen application parameters based on processing prescription data; comparing yield to soil zones based on processing soil data; etc.), where the personalized characterizations can be stored in association with corresponding user accounts (e.g., at a remote computing system for improving data storage and retrieval, such as for subsequent field test recommendations and/or other characterizations; etc.), presented to corresponding users in personalized reports, and/or otherwise processed. The technology can, however, provide any other suitable benefit(s) in the context of using non-generalized systems for identifying, characterizing, and/or recommending field tests, and/or for other suitable purposes.

### 3. System.

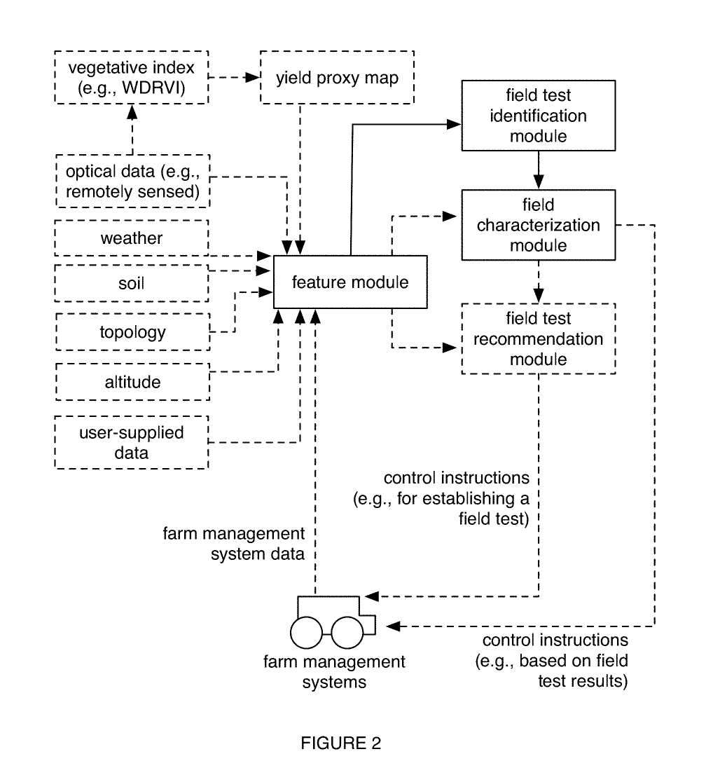

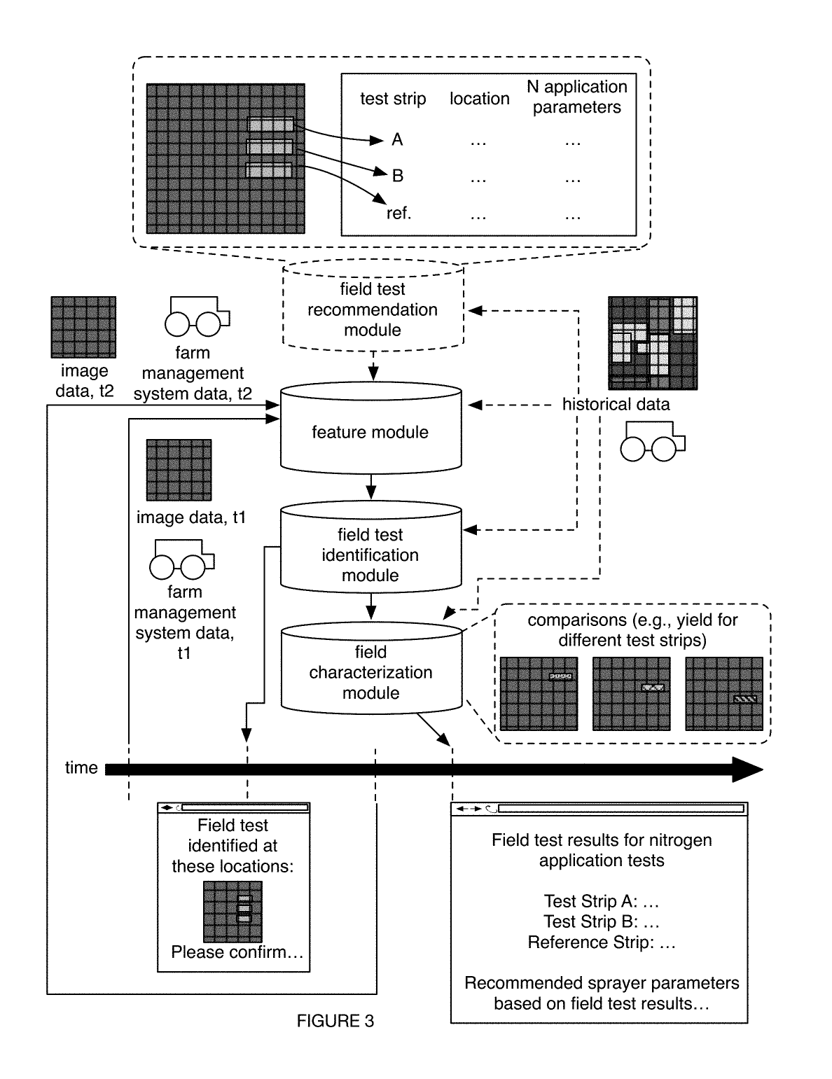

As shown in FIGS. 2-3, embodiments of a system for characterizing field tests can include: a feature module, a field test identification module, and/or a field characterization module. Additionally or alternatively, embodiments of the system can include: a field test recommendation module, field management systems (e.g., for the geographic region, including calendaring systems, farming activity logging systems, etc.), databases (e.g., databases including remotely sensed data, weather databases, soil databases, etc.), social networking systems, and/or any other suitable components. The system and/or components of the system (e.g., modules, databases, field management systems, etc.) can entirely or partially be executed by, hosted on, communicate with (e.g., wirelessly, through wired communication, etc.), and/or otherwise include: a remote computing system (e.g., servers, at least one networked computing system, stateless, stateful, etc.), a local computing system, vehicles, user devices, and/or by any suitable component. While the components of the system are generally described as distinct components, they can be physically and/or logically integrated in any manner. For example, the same or overlapping features and/or data types for determining field characteristics with the field characterization module can be used for determining field test recommendations with the field test recommendation module (e.g., and/or vice versa). Additionally or alternatively, any inputs and/or outputs associated with a given module can be leveraged as inputs into another module (e.g., in an ensemble approach, in a serial data generation approach performed according to computer-implemented rules, etc.). However, the functionality of the system can be distributed in any suitable manner amongst any suitable system components.

The feature module can function to determine features for identifying, characterizing, and/or recommending field tests. Field tests can be for field parameters including any of: seeding parameters, crop parameters (e.g., crop type, hybrid type, etc.), fertilizer parameters (e.g., prescriptions, fertilizer type, etc.), spraying parameters, equipment parameters (e.g., tillage parameters, harvesting parameters, etc.), environmental parameters (e.g., weather parameters, soil parameters, topological parameters, altitude parameters, etc.), location parameters, timing parameters, sampling parameters, parameters associated with field data, features, and/or other data described herein (e.g., module inputs, outputs, etc.), and/or any other suitable field parameters. Field tests can include any of: strip tests (e.g., with test strips, reference strips, etc.), geographic sub-region tests (e.g., sub-field level, etc.), geographic region tests (e.g., field level tests, cross-field tests, tests with adjacent geographic regions, with geographic regions in disparate locations, etc.) etc.), and/or field tests of any suitable scale (e.g., mapping to any suitable number of pixels of optical data, etc.). Field tests are preferably associated with a temporal indicator (e.g., ongoing during a time period; scheduled for a time period; etc.). In variations, field tests can include current field tests (e.g., ongoing field tests), historic field tests (e.g., completed field tests), future field tests (e.g., scheduled field tests), and/or field tests associated with any suitable temporal indicator, but can be alternatively time-independent (e.g., a generated, composite reference field test leveraged for comparisons to current field tests and/or recommended field tests, etc.).

Inputs used in generating, executing, and or updating the feature module (and/or any other suitable module) can include any one or more of: farm management system data (e.g., equipment sensor data; farm management system inputs and/or outputs; schedule of farm management activities; data associated with completed farm management activities; fertilizer prescription data; etc.), optical data (e.g., aerial satellite imagery; ground imagery; drone images; NAIP; rangefinder-based, such as LIDAR or TOF; satellite radar-based; optical data recorded in any suitable spectra; etc.), sampled data (e.g., field test sample data, non-field test sample data, etc.), weather data, soil data, topological data, altitude data, data extracted from physical soil samples, data collected from third party databases, user-inputted data, and/or any other suitable types of field data. Field data can be associated with any suitable geographic regions (e.g., a field, a field test region, etc.), temporal indicators, and/or other suitable aspects. geographically proximal regions, and/or any suitable geographic regions.

The feature module can output any one or more of: optical features, farm management system features (e.g., equipment features associated with tillage operations, spraying operations, seeding operations, harvesting operations, such as times of operation, locations of operation, operation type such as primary tillage versus secondary tillage, equipment sensor data, types of equipment, etc.), seeding features (e.g., based on the seed rate, seed prescription, seeding equipment capabilities, crop type, hybrid type, timing, location, etc.), fertilizer features (e.g., prescriptions, amount, type, application timing, location, etc.), growth stage features (e.g., prior crop growth stage; current crop growth stage; historic growth stage at a recurrent time point for a growing season; growing degree day values and/or other indexes; as received from a user; automatically estimated; etc.), crop density coefficients (e.g., for specific hybrids), natural features (e.g., weather parameters, soil parameters, topological parameters, historic vegetative performance values such as historic LAI, treatment history such as watering history and/or pesticide application history, disease history, pest history, crop anomaly history, etc.), yield features (e.g., current yield; historic yield; yield determined based on image data and/or non-image data; etc.), proxy map features (e.g., yield proxy map), altitude features, topological features, raw field data, field characteristics, field recommendation parameters, historic features, estimated features associated with a future temporal indicator, and/or any other suitable features associated with field tests.

For example, feature modules can process non-image data to generate feature sets including non-image features. In a specific example, farm management data (e.g., farm management system operation data such as sensor data, user inputs at the systems, other data collected at the systems; seed production records for the geographic regions; etc.) can be used as features and/or in generating features (e.g., as a training database for training field test identification modules, field characterization modules, and/or field test recommendation modules; etc.). In another example, image data can be used to generate feature sets including image features (e.g., shape features, color spectrum features, orientation features, object detection features, etc.).

Features can be generated for, compared across, and/or otherwise processed in relation to a plurality of: users (e.g., generating cross-user features from field data associated with a plurality of users); field tests (e.g., comparing a field test feature set for a first user to a field test feature set for a second user, such as for characterizing field tests based on parameters and/or results of other field tests example); geographic regions; and/or any other suitable components. The inputs (and/or outputs) for the feature module (and/or other suitable module) can be associated with the same time period (e.g., remotely sensed data of the field test-associated geographic region collected for May of a current year; farm management system sensor data collected for the same time period; data collected for the same growing season; such that the data associated with the same time period can be analyzed together and/or otherwise processed based on their temporal associations; etc.), be associated with different time periods, be associated with the same recurrent time period (e.g., feature values for every May, June, etc.), and/or be associated with any other suitable time periods that are related in any suitable manner The inputs are preferably automatically collected (e.g., from a third party service for remotely sensed optical data; received from communications by registered farm management systems; from a third-party database such as the Soil Survey Geography database; from user accounts associated with users; etc.), but can additionally or alternatively be manually collected (e.g., manually transmitting API requests to third party databases; manually requesting field data from users at an interface; etc.) and/or otherwise collected. However, the feature module can be configured in any suitable manner.

The field test identification module can function to identify (e.g., detect, classify, etc.) field tests and/or field test parameters. Inputs used in generating, executing, and/or updating the field test identification module (and/or any other suitable module) preferably include features associated with one or more feature modules (e.g., generated features, raw inputs, etc.), but can additionally or alternatively include any suitable types of data.

In a first variation, the field test identification module can use farm management system features as inputs. For example, the field test identification module can classify the presence of a field test at a geographic region based on equipment sensor data for the geographic region (e.g., where values, trends, and/or other suitable patterns of the equipment sensor data can indicate the presence of the field test). In one example, a large inter- or intra-row sensor deviation can be indicative of a test strip. In a specific example, sprayer data can indicate a fertilizer test strip (e.g., evaluating a fertilizer parameter) and a fertilizer reference strip (e.g., without the fertilizer parameter), where the field test identification module can identify the field test (e.g., the test strip and the reference strip) based on the sprayer data.

In a second variation, the field test identification module can use optical features as inputs. For example, the field test identification module can generate outputs based on optical features describing field patterns of a geographic region, where the field patterns can be correlated with field tests. Field patterns can include manmade patterns (e.g., equipment-induced field patterns; etc.), natural patterns (e.g., weather-based patterns; soil variation-based patterns; topological-based patterns; landscape-based patterns; etc.), patterns associated with any suitable time periods (e.g., patterns persisting over a plurality of growing seasons; a pattern generated during a current growing season, as indicated by a comparison between current and historic image data; etc.), and/or any other suitable types of patterns. In a specific example, the pattern identification module can compare field patterns of different geographic sub-regions in identifying a field test associated with a geographic sub-region of the geographic region (e.g., wherein the compared geographic sub-regions have similar natural patterns and/or expected natural performance; wherein a field test is identified as a smaller geographic sub-region that differs more than a threshold value from the reference sub-region). In another specific example, the field test identification module can use field performance values extracted from optical data of a geographic region (e.g., where relative performance values for geographic sub-regions can be indicative of a field test, such as differing more than a threshold value from the reference performance values), such as in a manner analogous to U.S. application Ser. No. 15/645,535 filed 10 Jul. 2017, which is herein incorporated in its entirety by this reference.

In a third variation, the field test identification module can use user-provided inputs (e.g., provided at web interface, provided at a mobile interface, etc.) indicating a field test. For example, the field test identification module can use farm management system data labeled by a user as corresponding to a field test (e.g., seeding data corresponding to seeding for a field test; application data corresponding to fertilizer application for the field test; etc.). In another example, a user can select the area of a field test from a graphical representation of a geographic region presented to the user. In another example, field parameters provided by a user (e.g., fertilizer application parameters, seeding parameters, etc.) can be used in inferring field tests (e.g., comparing fertilizer application rates across different geographic sub-regions to identify outliers indicating a test of an application parameter, etc.).

In a fourth variation, the field test identification module can use field test recommendation data as inputs. For example, field test recommendation parameters can be compared against farm management system features, optical features, and/or other suitable features to determine whether and/or to what degree a field test recommendation was followed. In another example, a user can provide feedback indicating whether a field test recommendation was followed. However, the field test identification module can use any suitable inputs in any suitable manner.

The field test identification module can output any one or more of: field test detections (e.g., presence of a field test; presence of a potential field test; probability of a field test being present; etc.); field test parameters (e.g., location parameters, such as coordinates, associated with a detected field test; field test types; temporal indicators associated with the field test; users associated with the field test; farm management systems associated with the field test; field parameters tested by a field test; etc.), and/or any other outputs associated with field tests.

Any number of field test identification modules (and/or other suitable modules) employing same or different approaches (e.g., algorithms; compared to other field test identification modules; compared to other types of modules; etc.) can be used. For example, different field test identification modules can be used based on data availability for a geographic region (e.g., using a convolutional neural network in response to availability of optical data; using a different machine learning model in response to availability of equipment sensor data; using a non-machine learning model in response to availability of user-provided inputs indicative of field tests; etc.). However, field test identification modules can be configured in any suitable manner.

The field characterization module can function to characterize one or more regions (e.g., geographic regions, geographic sub-regions, etc.) associated with a field test. Inputs used in generating, executing, and/or updating the field characterization module preferably include outputs of the field test identification module, but can additionally or alternatively include any suitable features (e.g., generated by the feature module) and/or any other suitable types of data. In an example, the field characterization module can characterize a field test (e.g., determine field performance values based on optical data; etc.) based on location parameters (e.g., coordinates; etc.) for the field test extracted from the field test identification module (e.g., where the optical data used for analysis can be selected based on the location parameters; etc.). In a second variation, the field characterization module can characterize a field test based on in-season data (e.g., imagery, such as remote imagery). The field characterization module can analyze a single trial (e.g., compare the current estimated yield or other field metric against a historic estimated yield value or field metric value for the same growing timeframe), analyze multiple trials (e.g., compare the field metric values for different concurrent trials), or analyze any other suitable set of trials.

In a first variation, the field characterization module can characterize a region associated with an identified field test independent of a field test recommendation (e.g., where a user placed a test strip in a manner independent from a field test recommendation, etc.). In a second variation, the field characterization module can characterize regions associated with a field test established based on a field test recommendation (e.g., as indicated by the field test identification module). For example, the field characterization module can leverage sample data collected according to sampling parameters defined in the field test recommendation. However, any suitable regions associated with field tests in any suitable manner (e.g., regions of the field test, regions adjacent the field test, reference regions, regions otherwise positionally related to the field test, regions with field parameters that can be used for comparison to field test regions; etc.) can be characterized.

The field characterization module can output field characteristics including: yield characteristics (e.g., in-season yield; end-of-season yield; yield proxies; field performance values; yield characteristics of improved accuracy based on historic field characteristics for historic field tests; predicted yield characteristics for other geographic regions based on field test results, such as through adjusting imagery calibration associated with imagery-based yield determination; etc.); field test comparisons (e.g., test strip comparisons; hybrid comparisons; yield comparisons for different sets of field parameters; etc.); application results (e.g., effects of variable rate application; of different fertilizer applications; etc.); field test robustness (e.g., indicating robustness of testing; time scale for appropriately evaluating the results of a field test; etc.); causes (e.g., of field characteristic values; of crop anomalies; manmade versus natural causes; etc.); recommendations (e.g., field parameters to apply to other geographic regions based on the results of the field test; field parameters to apply to geographic sub-regions independent from the region used for a field test; fertilizer prescriptions; farm management system operation parameters; etc.); crop anomalies (e.g., identifying anomalous geographic sub-regions sharing field parameters with those of field tests associated with results below a threshold; etc.); productivity zones; field pattern parameters (e.g., identifying causes of field patterns based on results of field tests; etc.); and/or any other suitable field-related characteristics. Field test comparisons can include comparisons of adjacent field passes (e.g., for prescription performance analysis), of non-adjacent field passes, of distinct field passes, and/or of regions of any suitable shape and size (e.g., regions including a test strip; regions including a reference strip; etc.). Yield characteristics and/or other suitable field characteristics can be determined for specific passes (e.g., using grid-based hashing for neighbor lookup; computing heading for pass comparisons; etc.) in evaluating field characteristics associated with field tests. Additionally or alternatively, the field characterization module can generate outputs in any suitable manner analogous to that described in U.S. application Ser. No. 15/483,062 filed 10 Apr. 2017, U.S. application Ser. No. 15/645,535 filed 10 Jul. 2017, and U.S. application Ser. No. 15/404,625 filed 12 Jan. 2017, each of which are herein incorporated in their entireties by this reference. However, the field characterization module can be configured in any suitable manner.

Additionally or alternatively, embodiments of the system can include a field test recommendation module, which can function to recommend a field test to a user. Inputs used in generating, executing, and/or updating the field characterization module preferably include outputs of the field characterization module (e.g., historical field characteristics associated with historical field tests, which can be used in identifying field parameters to be tested with a current field test recommendation; etc.), but can additionally or alternatively include any suitable features (e.g., generated by the feature module) and/or any other suitable types of data. In an example, a field test recommendation can account for variance in field parameters recommended for testing (e.g., accounting for background variance in manmade parameters, natural parameters; accounting for variance to optimize statistical power of the field test; etc.), such as based on historical data associated with the region recommended for the field test. In a specific example, a field test recommendation can include parameters for generating a comparison between a first region associated with high pH, eroded hills, and pooling water, and a second region without one or more of the characteristics. In another specific example, a field test recommendation can be used in evaluating productivity arising from a same soil type versus a plurality of soil types (e.g., based on soil maps indicating soil compositions at different locations; etc.).

In another example, a field test recommendation module can optimize a metric (e.g., the statistical power) of the trial, based on parameters of the available test sites (e.g., fields, field segments, etc.). The available test sites are preferably selected from the fields associated with (e.g., owned by, leased by, etc.) an agronomic entity (e.g., a farmer), but can be any suitable set of geographic regions. In a first specific example, the field recommendation module can iteratively: select a set of candidate geographic regions as test sites; determine the baseline variance for each of the selected candidate sites (e.g., variance in: soil type, aspect, rainfall, temperature, treatment practices, weed growth, pest occurrence, estimated yield, etc.); determine a metric for the selected candidate site combination; and select the candidate site combination with the highest or optimal metric value as the set of test sites to be used. The metric is preferably the statistical power of the trial, but can alternatively be: cost, speed, or be any other suitable metric. The baseline variance is preferably temporal variance for a given geographic region, but can additionally or alternatively be a spatial variance (e.g., across the geographic region, between the geographic region and an adjacent geographic region, etc.), or otherwise variant. The baseline variance can be determined from historical images (e.g., remote imagery, local imagery, etc.), elevation data, soil data (e.g., from a soil database), productivity estimates, or otherwise determined. In a second specific example, the field recommendation module can determine a set of desired test site characteristics that meet a given trial metric (e.g., statistical power); determine the characteristics (e.g., baseline variance) for each candidate site; and select the set of candidate sites that are most similar to the desired test site characteristics. However, differences in any suitable parameters can be evaluated according to a field test recommendation.

In variations, inputs into the field test recommendation module can include: yield characteristics (e.g., historic yield characteristics indicating field parameters to evaluate with a field test; predicted yield characteristics for field parameters included in a field test recommendation; yield characteristics for historic field tests; yield characteristics for regions independent from field tests; yield characteristics for the field tests; etc.); optical features (e.g., identifying location parameters for a field test recommendation based on current and/or historic field performance maps indicating field performance values and derived from optical data of the geographic region; etc.); farm management system features (e.g., historic equipment sensor data for farm management systems available to the user, which can be used in accounting for variability associated with outputs of the farm management systems, such as fertilizer application variability; historic fertilizer parameters, such as associated with historic applications of nitrogen, phosphorous, potassium; etc.); location parameters (e.g., historic field parameters associated with specific regions, etc.); natural features (e.g., weather parameters, topology parameters, elevation parameters; field size; soil delineations; soil maps of a geographic regions; etc.); target parameters (e.g., types of natural parameters to be experienced by the field test; target yield characteristics; target field characteristics; target profitability; etc.); and/or any other suitable types of inputs.

The field test recommendation module can output field test recommendations associated with any of: field test locations (e.g., coordinates; etc.); field test timing (e.g., beginning of field test; end of field test; etc.); field test scale (e.g., number of test strips, size of test strips, etc.), treatments (e.g., fertilizer types; fertilizer prescriptions; seeding prescriptions; crop type, such as hybrid type, corn with a specified genetic profile, etc.); operation parameters (e.g., tillage operations, spraying operations, seeding operations, equipment operations, etc.); sampling parameters (e.g., number of samples; locations for sampling; sampling time; guided sampling parameters; random sampling; composite sampling; grid sampling; etc.); other field parameters (e.g., value ranges for field parameters; etc.); estimated field test value (e.g., predicted profitability derivable from the field test; predicted return on investment from the field test; estimated cost; predicted yield; etc.); and/or any other suitable aspects. In an example, a field test recommendation can include location parameters for test strips predicted to experience similar natural parameters (e.g., which can account for natural variability in evaluating differences in a field parameter; etc.). In another example, sampling parameters for evaluating a nutrient stress can be determined based on optical data (e.g., in-season imagery; historical imagery; productivity zones identified based on optical data); fertilizer parameters; and/or suitable parameters associated with treatment performance. In another example, guided sampling parameters can be determined based on historic variability associated with the recommended region for the field test (e.g., using guided sampling for a low quality but large scale field tests; etc.).

Field test recommendations can be tailored for: geographic regions; geographic subregions (e.g., a recommendation including guidance for each 5 m² geographic sub-region of a geographic region; recommendations associated with any suitable resolution and granularity; etc.); users (e.g., based on user preferences associated with field parameters, such as user preferences for field test scale, cost, distance from a monitoring station, etc.); farm management systems (e.g., based on types of available farm management systems for implementing the recommendation; etc.); and/or any other suitable entities. However, field test recommendation modules can be configured in any suitable manner.

Modules and/or other components of the system can include: probabilistic properties, heuristic properties, deterministic properties, and/or any other suitable properties. The modules can applying any suitable processing operation (e.g., for image processing, feature extraction, etc.) including: image processing techniques (e.g., image filtering, image transformations, histograms, structural analysis, shape analysis, object tracking, motion analysis, feature detection, object detection, stitching, thresholding, image adjustments, etc.), data association (e.g., associating yield values with geographic locations; associating yield values with corresponding field test recommendation parameters; etc.), clustering, performing pattern recognition on data, fusing data from multiple sources, combination of values (e.g., averaging values, processing pixel values, etc.), compression, conversion, normalization, updating, ranking, validating, filtering (e.g., for baseline correction, data cropping, etc.), noise reduction, smoothing, filling (e.g., gap filling), aligning, model fitting, windowing, clipping, transformations, mathematical operations (e.g., derivatives, moving averages, summing, subtracting, multiplying, dividing, etc.), interpolating, extrapolating, visualizing, and/or any other suitable processing operations.

Modules and/or other components of the system can include and/or otherwise apply models associated with one or more of: classification (e.g., of presence of a field test; of types of field tests; etc.), decision-trees (e.g., for determining field test recommendations based on user preferences and/or field data a geographic region, etc.), regression (e.g., for predicting field characteristics associated with field test recommendations and/or different sets of field test parameters, etc.), neural networks (e.g., convolutional neural networks for processing remotely sensed optical data associated with field tests; etc.), heuristics, equations (e.g., weighted equations, etc.), selection (e.g., from a library, etc.), instance-based methods (e.g., nearest neighbor, etc.), regularization methods (e.g., ridge regression, etc.), Bayesian methods, kernel methods, supervised learning, semi-supervised learning, unsupervised learning, reinforcement learning, and/or any other approach. The module(s) can be run or updated: once; at a predetermined frequency; every time an instance of an embodiment of the method and/or subprocess is performed (e.g., sample collection according to sampling parameters determined for a field test recommendation, etc.); every time an unanticipated measurement value is received (e.g., indicating a crop anomaly, etc.); and/or at any other suitable frequency. Any number of instances of any suitable types of modules can be run or updated concurrently with one or more other modules, serially, at varying frequencies, and/or at any other suitable time.

The inputs and/or features (e.g., parameters used in an equation, features used in a machine learning model, etc.) used in modules can be determined through a sensitivity analysis, used for a plurality of modules (e.g., using image features and/or equipment features generated from the feature module as inputs for the field test identification module, the field characterization module, and the field recommendation module, etc.), received from other modules (e.g., outputs), received from a user account (e.g., from the user, from equipment associated with the user account, etc.), automatically retrieved (e.g., from an online database, received through a subscription to a data source, retrieved from a third party database, etc.), extracted from sampled sensor signals (e.g., remotely sensed images, sensor data from farm management systems, etc.), determined from a series of sensor signals (e.g., signal changes over time, signal patterns, etc.), and/or otherwise determined. Additionally or alternatively, inputs, outputs, and/or other suitable module-related aspects can include and/or be otherwise analogous to that described in U.S. application Ser. No. 15/483,062 filed 10 Apr. 2017 and U.S. application Ser. No. 15/645,535 filed 10 Jul. 2017, each of which are herein incorporated in their entireties by this reference. However, inputs, features, outputs, and/or other data associated with the modules can be configured in any suitable manner. The modules (and/or portions of the method 100) can be universally applicable (e.g., used across field tests, user accounts, geographic regions, etc.), specific to field tests (e.g., different modules used for different types of identified field tests, etc.), geographic regions (e.g., different modules used for different countries, for different coordinates associated with different weather parameters, etc.), user accounts (e.g., different modules used for different subgroups of users, etc.), crop type (e.g., corn, soy, alfalfa, wheat, rice, sugarcane, etc.), and/or can otherwise differ. However, the different system components can be configured in any suitable manner.

### 4. Method.

Identifying a field test for a geographic region S110 can function detect field tests, determine field test parameters, and/or otherwise identify field tests. In an example, identifying a field test can include determining correspondence between the field test and a field test recommendation (e.g., degree to which a field test recommendation is followed by a user; whether a field test recommendation was provided to the user; identifying the field test recommendation followed by the user; etc.). In another example, identifying a field test can include determining the scale of one or more field tests (e.g., number of test strips; field test size; number of parameters tested; ranges of parameter values; cost; etc.).

In another example, identifying a field test can include applying a field test identification module trained on a training dataset. In a specific example, generating the training dataset can include: establishing different known field tests (e.g., with known field parameters, etc.) using farm management systems; collecting farm management system data corresponding to the operations performed in establishing the known field tests; and training the field test identification module (e.g., machine learning model, etc.) using the farm management system data. In another specific example, generating the training dataset can include: collecting user-provided inputs indicative of the presence of field tests (e.g., prompting users to identify fields tests for their geographic regions, such as through prompting users for geocoordinates corresponding to the field test; etc.); collecting optical data (e.g., aerial satellite imagery, etc.) of geographic regions corresponding to the field tests; and training the field test identification module (e.g., classifier; convolutional neural network; etc.) using the optical data (e.g., pixel values, etc.). However, identifying field tests can be performed in any suitable manner. In another example, identifying a field test can include: receiving a set of crop types (e.g., crop hybrids) to be tested; determining a trial robustness metric (e.g., confidence interval, statistical power, etc.) for a given agronomic entity (e.g., for all fields belonging to a farmer, for unoccupied fields belonging to a farmer, etc.), and determining (e.g., retrieving) a set of optimal field conditions associated with each of the crop types, wherein the trial parameters (e.g., field characteristics, treatment parameters, etc.) can be selected to substantially match or optimize the statistical power of the agronomic trial.

Determining field characteristics for the field test based on field data S120 can function to characterize regions associated with the field test. Determining field characteristics can include accounting for variability (e.g., which can function to improve the accuracy of the field characteristics in identifying the effects of the tested parameters; etc.). In a first variation, determining field characteristics can include accounting for historic variability. In a specific example, historic variability can be determined using the method disclosed in U.S. application Ser. No. 15/645,535 filed 10 Jul. 2017, incorporated herein in its entirety by this reference, but can alternatively be determined using any other suitable method (e.g., soil database analysis, historic field measurement analysis, historic yield analysis, manually entered on a per-field or per-land unit basis, etc.). In a specific example, field characteristic values can be adjusted based on historical (and/or current) soil data, weather data, topological data, altitude data, predicted field characteristics (e.g., productivity estimates based on historical yield for the set of field parameters; other predicted field characteristics based on historical data; etc.), and/or any other suitable data. The field characteristics can be adjusted (e.g., by subtracting the field characteristic value resulting from natural influences, averaging the field characteristic values, etc.): after harvest, incrementally throughout the growing season (e.g., wherein the current field characteristic or crop status, as determined from optical analysis, is adjusted based on the historic field characteristic value or crop status of the corresponding historic recurrent time period), or at any suitable time. Adjusting the field characteristics can include: adjusting the absolute field characteristic value, adjusting the changes in the field characteristic value between recurrent time periods (e.g., subtracting the difference between a previous field test's characteristic value change from a first and second time and a historic characteristic value change between a first and second reference time, corresponding to the first and second time, from the next historic characteristic value change from the second time to a third time to account for in-season anomalies), or adjusting any other suitable value. In another specific example, yield characteristics can be adjusted to account for yield variability caused by natural influences (e.g., based on historic correlations between yield results and natural parameters; etc.) in order to evaluate manmade causes of the productivity results (e.g., tilling operations, harvesting operations, etc.) associated with a field test. In a second variation, determining field characteristics can include accounting for predicted variability (e.g., predicted weather parameters for the growing season, which can indicate the time required to evaluate the field test; etc.). In a third variation, determining field characteristics can include accounting for variability based on data for other geographic regions (e.g., associated with the user; associated with other users; etc.) independent of the field test. For example, actual field characteristics observed for geographic regions sharing a same or similar set of field parameters as that of a field test can be used in generating field characteristics for the field test. In a fourth variation, determining field characteristics can include classifying the field test. For example, the field test can be classified as less effective when the yield falls below the historic yield for the geographic sub-region. However, accounting for field variability can be performed in any suitable manner.

Determining field characteristics can include associating collected field data with other data. In a first variation, field data can be associated with identifiers (e.g., field test identifiers; user identifiers; geographic region identifiers; etc.), such as at a remote computing system. For example, collected optical data of a field test can be associated with a field test identifier identifying the field test. In a second variation, field data can be associated with field test recommendations (e.g., associating equipment sensor sample data with field test recommendation parameters, such as for facilitating comparisons between the recommendation parameters and the actual parameters of the field test for adjusting predicted field characteristics; etc.). In a third variation, field data can be tagged with descriptive parameters (e.g., location parameters identifying the location that the field data describes; temporal parameters; etc.) and/or any other suitable parameters. Additionally or alternatively, associating field data can be performed in any suitable manner. However, determining field characteristics can be performed in any suitable manner.

Additionally or alternatively, the method 100 can include providing field test recommendations S130 (e.g., through presentation at a web interface; through control instructions facilitating farm management system operation; through variable rate application instructions; etc.). Any number of field test recommendations can be provided. For example, providing field test recommendations can include providing a plurality of field test options for evaluating different sets of field parameters (e.g., evaluating different types of field parameters such as fertilizer application versus seeding parameters; evaluating different values of field parameters such as different fertilizer application amounts; etc.). In a specific example, field test options can be determined based on user preferences (e.g., for cost, for return on investment, for specific field parameters of interest, etc.). However, providing field test recommendations can be performed in any suitable manner.

Although omitted for conciseness, the embodiments include every combination and permutation of the various system components and the various method processes, including any variations, examples, and specific examples, where the method processes can be performed in any suitable order, sequentially or concurrently using any suitable system components. The system and method and embodiments thereof can be embodied and/or implemented at least in part as a machine configured to receive a computer-readable medium storing computer-readable instructions. The instructions are preferably executed by computer-executable components preferably integrated with the system. The computer-readable medium can be stored on any suitable computer-readable media such as RAMs, ROMs, flash memory, EEPROMs, optical devices (CD or DVD), hard drives, floppy drives, or any suitable device. The computer-executable component is preferably a general or application specific processor, but any suitable dedicated hardware or hardware/firmware combination device can alternatively or additionally execute the instructions. As a person skilled in the art will recognize from the previous detailed description and from the figures and claims, modifications and changes can be made to the embodiments without departing from the scope defined in the following claims.

We claim:

1. A method for guided management of an agronomic trial, comprising:
automatically determining a baseline variance for each of a set of geographic regions; and
determining a set of trial parameters for the agronomic trial by optimizing a statistical power of the agronomic trial based on the baseline variances of each of the set of geographic regions, wherein the set of agronomic trial parameters comprises a testing set of geographic regions selected from the set of geographic regions, wherein an agronomic machine is controlled based on the set of trial parameters.

2. The method of claim 1, wherein the baseline variance is determined based on historical remote imagery of each of the set of geographic regions.

3. The method of claim 1, wherein the set of geographic regions is associated with a single agronomic entity.

4. The method of claim 1, wherein the set of trial parameters further comprises a set of sampling locations within the testing set of geographic regions, wherein the sampling locations are selected based on the baseline variance.

5. The method of claim 1, wherein the baseline variance for each geographic region comprises at least one of: a baseline variance of soil across the geographic region, rainfall for the geographic region, and temperatures for the geographic region.
